(19) United States
(12) Patent Application Publication
(10) Pub. No.: US 2015/0306178 A1
(43) Pub. Date: Oct. 29, 2015

(54) METHODS AND COMPOSITIONS FOR TREATING AND PREVENTING TINNITUS

(71) Applicant: Auris Medical AG, Basel (CH)

(72) Inventor: Thomas MEYER, Zuchwil (CH)

(21) Appl. No.: 14/694,122

(22) Filed: Apr. 23, 2015

Related U.S. Application Data
(60) Provisional application No. 61/983,394, filed on Apr. 23, 2014.

(51) Int. Cl. A61K 38/17; A61K 38/10; A61K 47/36; C07K 7/08; C07K 14/47

(52) U.S. Cl. CPC A61K 38/1709; C07K 7/08; C07K 14/47; A61K 47/36; A61K 38/10

(57) ABSTRACT

The invention relates to pharmaceutical compositions and methods for treating inner ear disorders. In particular, the invention provides a method for treating and/or preventing acute inner ear tinnitus in a subject in need thereof by administering a pharmaceutical composition comprising a peptide inhibitor of c-Jun N-terminal kinase.

### CROSS-REFERENCE TO RELATED APPLICATIONS

The present application claims priority to, and the benefit of U.S. Provisional Patent Application Ser. No. 61/983,394, filed on Apr. 23, 2014, which is herein incorporated by reference in its entirety for all purposes.

### FIELD OF THE INVENTION

The invention relates to the fields of otology and neurotology. In particular, the invention relates to pharmaceutical compositions and methods for ameliorating, treating, and/or preventing inner ear disorders, such as acute inner ear tinnitus. The pharmaceutical compositions may comprise a peptide inhibitor of c-Jun N-terminal kinase (JNK).

### BACKGROUND OF THE INVENTION

Tinnitus, the perception of sound without external acoustic stimulation, is a very common disorder. According to some recent estimates, approximately 25% of American adults have experienced tinnitus, with nearly 8% having frequent occurrences (Shargorodsky et al., 2010). European population studies estimate that 7-14% of the population has spoken to their doctor about tinnitus, while potentially disabling tinnitus occurs in 1-2.4% of people (Vesterager, 1997). Tinnitus may seriously impact the ability to sleep or relax, or lead to tiredness, irritation, nervousness, despair, frustration or depression (Chan, 2009; Stouffer et al., 1990).

Many tinnitus patients are prepared to try a wide variety of treatments in the search for effective relief (Tyler, 2012). While approaches such as tinnitus retraining therapy (Jastreboff, 2007) or cognitive behavioural therapy (Cima et al., 2012) may provide relief for certain patients, there exists no universal standard of care for tinnitus or approved tinnitus drug (Langguth et al., 2012), provoking substantial frustration among patients and physicians (Hall et al., 2011). Thus, there is a need in the art for additional therapeutic agents that can effectively ameliorate or prevent the development of tinnitus associated with various conditions.

### SUMMARY OF THE INVENTION

The present invention provides a method of ameliorating or reducing the occurrence of tinnitus (e.g. acute inner ear tinnitus) induced by a cochlear insult in a subject in need thereof. In one embodiment, the method comprises administering to the subject a pharmaceutical composition comprising a therapeutically effective amount of a peptide inhibitor of JNK or a pharmaceutically acceptable salt thereof. The tinnitus to be ameliorated, treated, suppressed, and/or prevented may be acute, subacute, or chronic. In particular embodiments, the subject is human.

In some embodiments, the tinnitus to be ameliorated, treated, suppressed, and/or prevented is induced by a cochlear insult selected from acute acoustic trauma, presbycusis, ischemia, anoxia, barotrauma, otitis media, exposure to ototoxic drugs, conductive hearing loss, or sudden deafness. In one embodiment, the tinnitus is associated with hearing loss. The hearing loss may, in certain embodiments, be severe or profound hearing loss (e.g. a hearing loss of at least 60 dB within 48 hours of onset). In some embodiments, the JNK peptide inhibitor is administered to the subject at the time of an event that could potentially induce a cochlear insult or shortly thereafter. For instance, in one embodiment, the JNK peptide inhibitor is administered to the subject within a few days to one week following an event inducing a cochlear insult.

The JNK peptide inhibitors used in the pharmaceutical compositions and methods of the invention are generally peptides of no more than 50 amino acids in length and comprise an amino acid sequence corresponding to a JNK binding domain from c-Jun proteins or JNK-interacting proteins (JIPs), e.g. JIP-1 (a.k.a. islet-brain protein 1), JIP-2 (a.k.a. islet-brain protein 2), and JIP-3. For instance, in some embodiments, the JNK peptide inhibitors comprise an amino acid sequence with substantial sequence homology to a sequence of SEQ ID NOs: 1-4 and 13-45. In certain embodiments, all chiral amino acids in the JNK peptide inhibitor are in the D configuration. In other embodiments, all chiral amino acids in the JNK peptide inhibitor are in the L configuration. In one embodiment, the JNK peptide inhibitor comprises or consists of a sequence of SEQ ID NO: 2 or SEQ ID NO: 3. In another embodiment, the JNK peptide inhibitor comprises or consists of a sequence of SEQ ID NO: 14 or SEQ ID NO: 16. In still another embodiment, the JNK peptide inhibitor comprises or consists of a sequence of SEQ ID NO: 18 or SEQ ID NO: 20.

The pharmaceutical compositions comprising a JNK peptide inhibitor may be formulated as a gel. In some such embodiments, the pharmaceutical compositions comprise hyaluronic acid. The pharmaceutical compositions can be administered topically to the subject, for example, via the round window membrane or the oval window membrane to the inner ear. In certain embodiments, the pharmaceutical composition comprising the JNK peptide inhibitor is delivered to the middle ear. In other embodiments, the pharmaceutical composition comprising the JNK peptide inhibitor is administered to the subject by an intratympanic injection.

### DETAILED DESCRIPTION OF THE INVENTION

The present invention is based, in part, on the discovery that inhibition of c-Jun N-terminal kinase (JNK) in cells of the cochlea reduces the loudness of tinnitus and leads to more frequent occurrences of complete remission of the tinnitus, particularly in patients having severe hearing loss.

INK is a member of the stress-activated group of mitogen-activated protein kinases involved in apoptosis following extracellular stress insults and inflammation (Manning, 2003). Its inhibition prevents formation of transcription complexes and further progress along the apoptotic pathway or activation of genes which encode inflammatory molecules. The JNK signaling pathway has been implicated in the apoptosis of injured cells in the cochlea (e.g. hair cells and spiral ganglion neurons) following traumatic injury or cochlear inflammation (Zine et al., 2004; Abi-Hachem et al., 2010; Hu et al., 2002; Ma et al., 2000; Barkdull et al., 2007). Inhibition of JNK has been reported to be otoprotective in various models of cochlear insult and protective against hearing loss (Wang et al., 2003; Wang et al. 2007; and Coleman et al. 2007; Grindal et al., 2010; and Eshraghi et al., 2011). However, the role of the JNK signaling cascade in the development of tinnitus is not well understood.

The present inventor has surprisingly found that inhibition of JNK with a peptide inhibitor can reduce the severity and occurrence of tinnitus induced by a cochlear insult, including tinnitus associated with acute acoustic trauma or idiopathic sudden sensorineural hearing loss. Without being bound by theory, it is believed that inhibition of JNK with the peptide inhibitors described herein protect the morphology of cochlear sensory cells (e.g., inner hair cells or spiral ganglion neurons) thereby preventing them from generating aberrant excitation of auditory nerve fibers resulting in the perception of tinnitus. Unlike agents that are designed to reduce or prevent aberrant activity of glutamate receptors or to restore the excitatory-inhibitory balance following aberrant activity triggered by damaged sensory cells (e.g. following glutamate excitotoxicity), the JNK peptide inhibitors protect the sensory cells from permanent damage in the first place so that they do not trigger such aberrant activity. Accordingly, the present invention provides a method of ameliorating or reducing the occurrence of acute inner ear tinnitus induced by a cochlear insult in a human in need thereof. In one embodiment, the method comprises administering to the human a pharmaceutical composition comprising a therapeutically effective amount of a peptide inhibitor of JNK or a pharmaceutically acceptable salt thereof.

As used herein, “ameliorating tinnitus” refers to the improvement of one or more aspects of tinnitus experienced by the patient following administration of a pharmaceutical composition of the invention. For example, tinnitus in a patient would be considered to be ameliorated if the loudness of the tinnitus, frequency of the tinnitus, and/or duration of the tinnitus perceived by the patient is reduced or the tinnitus is completely resolved. Methods of assessing the severity and presence of tinnitus in a patient are known to those of skill in the art and can include, but are not limited to, validated psychometric questionnaires, such as the Tinnitus Handicap Inventory, the Tinnitus Reaction Questionnaire, and the Tinnitus Functional Index. See, e.g., Figueiredo et al., 2009; Kamalski et al., 2010; and Meikle et al., 2011, each of which is herein incorporated by reference in its entirety.

In some embodiments, the tinnitus to be ameliorated, treated, and/or prevented is induced by a cochlear insult. The cochlear insult may cause damage to or modify the activity of cells in the cochlea, including the outer hair cells, the inner hair cells, and spiral ganglion neurons. The cochlear insult may result from acute acoustic trauma, presbycusis, ischemia, anoxia, barotrauma, otitis media, exposure to ototoxic drugs, conductive hearing loss, or sudden deafness. The tinnitus to be ameliorated, treated, and/or prevented with the methods of the invention may be acute, subacute, or chronic.

As used herein, the term “ototoxic drug” refers to any compound characterized by having a deleterious effect upon either the eighth nerve or upon the organs of hearing and balance. Such ototoxic drugs, which can produce tinnitus as a side effect, include, but are not limited to, aminoglycoside antibiotics, anti-inflammatories, sedatives, antidepressants, quinine medications, and chemotherapeutic agents (e.g., cisplatin).

In certain embodiments, the tinnitus to be ameliorated, treated, and/or prevented is associated with hearing loss. Thus, in some embodiments, the subject administered with a pharmaceutical composition of the invention has or is diagnosed with hearing loss. In one embodiment, the hearing loss is acute sensorineural hearing loss. In another embodiment, the acute sensorineural hearing loss is induced by acute acoustic trauma. In still another embodiment, the hearing loss is idiopathic sudden sensorineural hearing loss. In particular embodiments, the subject administered with a pharmaceutical composition of the invention has or is diagnosed with severe or profound hearing loss. As used herein, “severe or profound hearing loss” refers to a loss of hearing of at least 60 dB within 48 hours of onset as measured by standard techniques known in the art. One exemplary method for evaluating the severity of hearing loss in a subject is described in Example 1. In some embodiments, a subject with severe or profound hearing loss has or is diagnosed with a hearing loss of at least 60 dB within 48 hours of onset. The present inventor has found that the pharmaceutical compositions described herein are particularly useful in ameliorating or reducing the occurrence of tinnitus in subjects having a hearing loss of at least 60 dB within 48 hours of onset. In other embodiments, the subject administered with a pharmaceutical composition of the invention has or is diagnosed with moderate acute hearing loss. As used herein, “moderate hearing loss” refers to acute loss of hearing of at least 40 dB that persists after 48 hours of onset.

In some embodiments of the methods of the invention, the pharmaceutical composition comprising the peptide inhibitor of JNK is administered to the subject within four weeks following the cochlear insult from which the tinnitus arises. In other embodiments, the pharmaceutical composition is administered to the subject within two weeks following the cochlear insult inducing the tinnitus. In still other embodiments, the pharmaceutical composition is administered to the subject within one week following the cochlear insult inducing the tinnitus. In certain preferred embodiments, the pharmaceutical composition is administered to the subject within three to five days following the cochlear insult. For instance, in one particular embodiment, the pharmaceutical composition is administered to the subject about three days following the cochlear insult. In certain other embodiments, the subject is administered the pharmaceutical composition within two days following the cochlear insult inducing the tinnitus.

In other embodiments, the pharmaceutical compositions of the invention are administered to a subject as a prophylactic measure to reduce the development of tinnitus. For instance, in one embodiment, the pharmaceutical composition is administered to a subject prior to or during the subject's exposure to a potential cochlear insult. By way of example, a pharmaceutical composition of the invention may be administered to a subject prior to exposure to ototoxic drugs or excessively loud noises.

In certain embodiments, the peptide inhibitor of JNK to be used in the pharmaceutical compositions and methods of the invention comprises an amino acid sequence derived from the JNK binding domain of islet-brain 1 protein or islet-brain 2 protein, which are also known as JNK-interacting protein (JIP) 1 and 2, respectively. See Bonny et al., 2001, which is hereby incorporated by reference in its entirety. The JIP family of proteins has been shown to function as scaffold proteins in JNK signaling cascades. See Weston and Davis, Science 292: 2439-2440, 2001. For example, in such embodiments, the JNK peptide inhibitor comprises or consists of an amino acid sequence with substantial sequence homology to a sequence of DQSRPVQPFLNLTTPRKPR (SEQ ID NO: 1), RPKRPTTLNLFPQVPRSQD (SEQ ID NO: 4), DTYRPKRPTTLNLFPQVPRSQDT (SEQ ID NO: 13), TDQSRPVQPFLNLTTPRKPRYTD (SEQ ID NO: 15), HKHRPTTLRLTTLGAQDS (SEQ ID NO: 17), SDQAGLTTLRLTTPRHKH (SEQ ID NO: 19), RPKRPTTLNLF (SEQ ID NO: 21), or FLNLTTPRKPR (SEQ ID NO: 23). In other embodiments, the JNK peptide inhibitor comprises or consists of an amino acid sequence with substantial sequence homology to a sequence of RPKRPKTLNLF (SEQ ID NO: 25), FLNLTKPRKPR (SEQ ID NO: 27), RPKRPTFLNLF (SEQ ID NO: 29), FLNLFTPRKPR (SEQ ID NO: 31), RPKRPTSLNLF (SEQ ID NO: 33), FLNLSTPRKPR (SEQ ID NO: 35), RPKRPTTLNLD (SEQ ID NO: 37), DLNLTTPRKPR (SEQ ID NO: 39), PKRPTTLNLF (SEQ ID NO: 41), or FLNLTTPRKP (SEQ ID NO: 43). In some embodiments, the JNK peptide inhibitor comprises an amino acid sequence derived from the JNK binding domain of (JNK)-interacting protein-3 (JIP3) (Genbank Accession #NP_{—}055948.2). In certain embodiments, the JNK peptide inhibitor comprises or consists of a sequence selected from SEQ ID NO: 1, 4, 13, 15, 17, 19, 21, 23, 25, 27, 29, 31, 33, 35, 37, 39, 41, and 43.

The JNK inhibitor peptides can also be derived from c-Jun proteins. For example, a synthetic peptide comprising the JNK binding region on c-Jun, which corresponds to amino acids 33-79, is described in U.S. Pat. No. 6,514,745 as a competitive inhibitor of the naturally occurring c-Jun to decrease the amount of c-Jun activation by JNK. A cell-permeable 37-mer peptide consisting of the human c-Jun 6 domain (amino acids 33-57) sequence and the HIV-TAT protein transduction domain (amino acids 47-57), fused by a γ-aminobutyric acid (GABA) spacer (e.g., Ac-YGRKKRRQRRR-gaba-ILKQSMTLNLADPVGSLKPHLRAKN-NH2 (SEQ ID NO: 45)) was shown to specifically disrupt c-Jun/JNK complex formation and the subsequent phosphorylation and activation of c-Jun by JNK both in vitro and in intact cells. See Holzberg et al., J Biol Chem. 278(41):40213-23, 2003. Thus, in one embodiment, the JNK inhibitor used in the pharmaceutical compositions and methods of the invention comprises or consists of the SEQ ID NO: 45.

In some embodiments, the JNK inhibitor peptide binds to JNK. In other embodiments the JNK peptide inhibitor inhibits the activation of one or more components of the JNK signaling cascade, such as activation of a transcription factor, e.g. c-Jun, ATF-2, ELK-1, or p53. Other suitable JNK peptide inhibitors that may be used in the pharmaceutical compositions and methods of the invention are those described in U.S. Pat. Nos. 6,410,693; 6,610,820; 8,236,924; 8,080,517; and 8,183,339, each of which is hereby incorporated by reference in its entirety.

JNK peptide inhibitors comprising the amino acid sequences described herein may be about 15, about 20, about 25, about 30, about 35, about 40, about 45, about 50 or more amino acids. In some embodiments, the JNK peptide inhibitors comprise no more than 50 amino acids. In other embodiments, the JNK peptide inhibitors comprise no more than 35 amino acids. In certain embodiments, the JNK peptide inhibitors comprise from about 20 amino acids to about 50 amino acids or from about 25 amino acids to about 40 amino acids.

The JNK inhibitor peptides can be polymers of L-amino acids, D-amino acids, or a combination of both. For example, in some embodiments, the peptides are D retro-inverso peptides. The term “retro-inverso isomer” refers to an isomer of a linear peptide in which the direction of the sequence is reversed, the term “D-retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into a D retro-inverso peptide by synthesizing a reverse of the sequence for the corresponding native L-amino acid sequence.

In some embodiments, the JNK peptide inhibitors comprise an amino acid sequence in which all of the chiral amino acids are in the D configuration. In other embodiments, the JNK peptide inhibitors comprise an amino acid sequence in which all of the chiral amino acids are in the L configuration. All amino acids except glycine can occur in two isomeric forms, because of the possibility of forming two different enantiomers around the central carbon atom. Thus, “chiral amino acids” refer to amino acids that have four different substituents attached to the central carbon atom.

The JNK peptide inhibitors that can be used in the pharmaceutical compositions and methods of the present invention further include derivatives, fragments, homologs, analogs and conservative variants of JNK inhibitor peptides herein described. As used herein, a conservative variant refers to an alteration in the amino acid sequence that does not adversely affect the biological functions of the peptide. A substitution, insertion or deletion is said to adversely affect the peptide when the altered sequence prevents or disrupts a biological function associated with the peptide. For example, the overall charge, structure or hydrophobic/hydrophilic properties of the peptide may be altered without adversely affecting a biological activity. Accordingly, the amino acid sequence can be altered, for example to render the peptide more hydrophobic or hydrophilic, without adversely affecting the biological activities of the peptide.

Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g. in sequence and in function with a protein having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. In some embodiments, substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. In some embodiments, substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

Derivatives, fragments, and analogs of the peptide inhibitors described herein are defined as sequences of at least 4 contiguous amino acids, a length sufficient to allow for specific recognition of an epitope. The length of the fragments is less than the length of the corresponding full-length polypeptide from which the JNK inhibitor peptide is derived. Derivatives and analogs may be full length or other than full length, if the derivative or analog contains a modified nucleic acid or amino acid. Derivatives or analogs of the JNK inhibitor peptides include, e.g., molecules including regions that are substantially homologous to the peptides, in some embodiments, by at least about 30%, 50%, 70%, 80%, or 95%, 98%, or even 99%, identity over an amino acid sequence of identical size or when compared to an aligned sequence in which the alignment is done by a computer homology program known in the art. For example sequence identity can be measured using sequence analysis software (Sequence Analysis Software Package of the Genetics Computer Group, University of Wisconsin Biotechnology Center, 1710 University Avenue, Madison, Wis. 53705), with the default parameters therein. In one embodiment, the JNK peptide inhibitors comprise a sequence that is at least 80% identical to any one of SEQ ID NOs: 1 to 4 and 13-45. In another embodiment, the JNK peptide inhibitors comprise a sequence that is at least 90% identical to any one of SEQ ID NOs: 1 to 4 and 13-45. In still another embodiment, the JNK peptide inhibitors comprise a sequence that is at least 95% identical to any one of SEQ ID NOs: 1 to 4 and 13-45.

Where a particular polypeptide is said to have a specific percent identity to a reference polypeptide of a defined length, the percent identity is relative to the reference peptide. Thus, by way of example, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria.

Another variation of the JNK peptide inhibitors is the linking of from one to fifteen amino acids or amino acid analogs to the N-terminal or C-terminal amino acid of the JNK peptide inhibitors described herein. Analogs of the JNK peptide inhibitors can be prepared by adding from one to fifteen additional amino acids to the N-terminal, C-terminal, or both N- and C-terminals, of an active peptide inhibitor, where such amino acid additions do not adversely affect the ability of the peptide to bind to JNK.

JNK-inhibitor peptides are obtained or produced by methods well-known in the art, e.g. chemical synthesis or genetic engineering methods. For example, a INK peptide inhibitor, including a desired region or domain, may be synthesized by use of a peptide synthesizer. Alternatively, a JNK peptide inhibitor can be synthesized by recombinant expression by inserting a vector encoding the INK peptide inhibitor into an appropriate host cell and culturing the host cell under conditions to promote expression. Suitable host cells include, but are not limited to, mammalian cells, insect cells, yeast cells, and bacteria cells. The JNK peptide inhibitors can also be synthesized using cell-free translation systems known in the art.

In certain embodiments, the JNK peptide inhibitors are chimeric peptides comprising a JNK binding domain fused to a protein transduction domain (PTD). PTDs are heterogeneous in size and lack sequence homology, although most share a positive charge and are amphipathic. In certain embodiments, PTDs can be antimicrobial peptides such as protegrin 1, Bactenecin 7, Buforin, and Maginin; a host of arginine-rich RNA- and DNA-binding peptides (e.g., HIV-1 transactivating protein (TAT) and Drosophila homeodomain transcription factor Antennapedia (a.k.a. Penetratin); chimeric PTDs such as Transportan; lysine- and arginine-rich peptides derived from phage-display libraries; polyarginine; and most recently, β-homolysine oligomers. See Fisher et al., 2001; Lindsay, 2002; Tung et al., 2002; Bogoyevitch et al., 2002; and Garcia-Echeverria et al., 2003, each of which is hereby incorporated by reference in its entirety. In certain embodiments, the PTDs are reverso-, retro-inverso, and enantio-forms of any of the PTDs described herein. Exemplary PTDs that may be fused to JNK-binding domains include PTDs derived from HIV TAT protein (e.g. GRKKRRQRRRPP (SEQ ID NO: 5) or PPRRRQRRKKRG (SEQ ID NO: 6)), Antennapedia protein (e.g. RQIKIWFQNRRMKWKK (SEQ ID NO: 7) or RRMKWKK (SEQ ID NO: 8)), SynB1 (e.g. RGGRLSYSRRRFSTSTGR (SEQ ID NO: 9)), SynB3 (RRLSYSRRRF (SEQ ID NO: 10)), SynB5 (RGGRLAYLRRRWAVLGR (SEQ ID NO: 11)) or polyarginine (RRRRRRRR (SEQ ID NO: 12)). The PTD sequence can be fused to the N-terminus or C-terminus of the JNK-binding domain peptide. A linker of 1 to 10 amino acids can be inserted between the PTD sequence and the JNK-binding domain sequence. In some embodiments, a linker of two proline residues is used.

In particular embodiments, the PTD fused to the JNK-binding domain is derived from the TAT protein. In such embodiments, the chimeric peptides may comprise or consist of a sequence of:

(SEQ ID NO: 2)
DQSRPVQPFLNLTTPRKPRPPRRRQRRKKRG;
(SEQ ID NO: 3)
GRKKRRQRRRPPRPKRPTTLNLFPQVPRSQD;
(SEQ ID NO: 14)
GRKKRRQRRRPPDTYRPKRPTTLNLFPQVPRSQDT;
(SEQ ID NO: 16)
TDQSRPVQPFLNLTTPRKPRYTDPPRRRQRRKKRG;
(SEQ ID NO: 18)
GRKKRRQRRRPPHKHRPTTLRLTTLGAQDS;
(SEQ ID NO: 20)
SDQAGLTTLRLTTPRHKHPPRRRQRRKKRG;
(SEQ ID NO: 22)
GRKKRRQRRRPPRPKRPTTLNLF;
(SEQ ID NO: 24)
FLNLTTPRKPRPPRRRQRRKKRG;
(SEQ ID NO: 26)
GRKKRRQRRRPPRPKRPKTLNLF;
(SEQ ID NO: 28)
FLNLTKPRKPRPPRRRQRRKKRG;
(SEQ ID NO: 30)
GRKKRRQRRRPPRPKRPTFLNLF;
(SEQ ID NO: 32)
FLNLFTPRKPRPPRRRQRRKKRG;
(SEQ ID NO: 34)
GRKKRRQRRRPPRPKRPTSLNLF;
(SEQ ID NO: 36)
FLNLSTPRKPRPPRRRQRRKKRG;
(SEQ ID NO: 38)
GRKKRRQRRRPPRPKRPTTLNLD;
(SEQ ID NO: 40)
DLNLTTPRKPRPPRRRQRRKKRG;
(SEQ ID NO: 42)
GRKKRRQRRRPPPKRPTTLNLF;
or
(SEQ ID NO: 44)
FLNLTTPRKPPPRRRQRRKKRG.

The pharmaceutical compositions to be employed in the methods of the invention comprise a therapeutically effective amount of a JNK peptide inhibitor or a pharmaceutically acceptable salt thereof and a pharmaceutically acceptable carrier or excipient. JNK peptide inhibitors included in the pharmaceutical compositions of the present invention can be in free form or the form of a salt, where the salt is pharmaceutically acceptable. Examples of such a pharmaceutically acceptable salt include, but are not limited to, those formed with organic acids (e.g. acetic, lactic, citric, malic, formaric, tartaric, stearic, ascorbic, succinic, benzoic, methanesulfonic, toluenesulfonic, or pamoic acid), inorganic acids (e.g., hydrochloridic, nitric, diphosphoric, sulphuric, or phosphoric acid), and polymeric acids (e.g., tannic acid, carboxymethyl cellulose, polylactic, polyglycolic, or copolymers of polylactic-glycolic acids). In one particular embodiment, the JNK peptide inhibitor is present in the pharmaceutical composition as an acetate salt.

Pharmaceutical compositions for any route of administration of this invention contain a therapeutically effective amount of the JNK peptide inhibitor, and, as may be necessary, inorganic or organic, solid or liquid pharmaceutically acceptable carriers or excipients. Pharmaceutical compositions suited for topical administration to the inner ear include aqueous solutions or suspensions, which, e.g. in the case of lyophilized formulations that contain the active ingredient alone or together with a carrier, may be prepared prior to use. They further include gels, which may be biodegradable or non-biodegradable, aqueous or non-aqueous, or microsphere based. Examples of gel-forming biocompatible polymers include, but are not limited to, hyaluronic acid resp. hyaluronates, lecithin gels, (poly)alanine derivatives, pluronics, poly(ethyleneglycol), poloxamers, chitosans, xyloglucans, collagens, fibrins, polyesters, poly(lactides), poly(glycolide) or their co-polymers PLGA, sucrose acetate isobutyrate, and glycerol monooleate. Preferred are gels which can be easily administered into the middle ear, release the peptide inhibitor over an extended period of time, and allow for a high percentage of the peptide inhibitor to be delivered into the inner ear.

Hyaluronic acid, which is preferably used as the biocompatible polymer in the pharmaceutical composition of the present invention, is a physiological substance that is widely distributed in the extracellular matrix of connective tissues in all organs of the body. It occurs in various molecular weights and is reported to be non-antigenic. Moreover, it has an excellent biocompatibility and is also biodegradable. Hyaluronic acid is a naturally occurring polysaccharide, a glycosaminoglycan composed of a long-chain polymer containing repeating disaccharide units of sodium glycuronate-N-acetylglucosamine. The main properties of hyaluronic acid are that it binds water and hence forms a degradable gel with high viscosity. The viscosity of the hyaluronic acid solutions increases with concentration and molecular weight. The JNK peptide inhibitors can be either dissolved or suspended in the hyaluronic acid gel. In some embodiments, the pharmaceutical compositions comprise about 0.5% to about 1% of hyaluronic acid. In other embodiments, the pharmaceutical compositions comprise about 0.7% to about 0.9% of hyaluronic acid.

The pharmaceutical compositions may be sterilized and/or may contain adjuvants, e.g. preservatives, stabilizers, wetting agents and/or emulsifiers, salts for regulating the osmotic pressure and/or buffers. In some embodiments, the pharmaceutical compositions comprise a buffer that buffers the pH of the composition from about 6.0 to about 7.4. In certain embodiments, the pharmaceutical compositions comprise a phosphate buffer. In related embodiments, the phosphate buffer buffers the pH of the composition to about 6.2.

The pharmaceutical compositions of the invention may, if desired, contain further pharmacologically active substances or other components, such as antibiotics, e.g., fluoroquinolones, anti-inflammatory agents, e.g., steroids, cortisone, analgesics, antipyrine, benzocaine, procaine, etc. The pharmaceutical compositions may be prepared by any of the methods well known in the art of pharmacy, e.g. by conventional mixing, granulating, confectioning, dissolving or lyophilizing methods, and contain from about 0.01 to 100%, preferably from about 0.1 to 50% (lyophilisates up to 100%), of active ingredient.

The pharmaceutical compositions comprising a JNK peptide inhibitor can be administered to a subject orally, intravenously, subcutaneously, intraperitoneally, intramuscularly, rectally or topically. In certain embodiments, topical administration to the inner ear is preferred, as therapeutically effective doses with systemic administration may induce undesired side effects. The only requirement for administration in the present invention is that a therapeutically effective amount of a pharmaceutical composition comprising a JNK peptide inhibitor be able to reach cochlear cells in the afflicted individual.

Administration of the pharmaceutical composition to the inner ear may be accomplished by various delivery techniques. Such techniques include the use of devices or drug carriers to transport and/or deliver the JNK peptide inhibitor in a targeted fashion to the membranes of the round or oval window, where it diffuses into the inner ear or is actively infused. Examples are otowicks (see e.g. U.S. Pat. No. 6,120,484 to Silverstein, incorporated herein by reference), round window catheters (see e.g. U.S. Pat. Nos. 5,421,818; 5,474,529; 5,476,446; 6,045,528; all to Arenberg, or U.S. Pat. No. 6,377,849 and U.S. Patent Publication No. 2002/0082554 to Lenarz, all of which are incorporated herein by reference), microimplants (see e.g. WO2004/064912 by Jukarainen et al.) or various types of gels, foams, fibrins or other drug carriers, which are placed in the round window niche or on the oval window, and loaded with the compound for sustained release (see e.g. WO 97/38698 by Manning; Silverstein et al., Otolaryngology—Head and Neck Surgery 120 (5): 649-655 (1999); Balough et al., Otolaryngology—Head and Neck Surgery 119 (5): 427-431 (1998), each of which is hereby incorporated by reference in its entirety). Other suitable delivery techniques include the use of devices which are inserted into the cochlear duct or any other part of the cochlea (see e.g. U.S. Pat. No. 6,309,410 to Kuzma, incorporated herein by reference). The pharmaceutical composition comprising a JNK peptide inhibitor may also be administered to the inner ear by intratympanic injection, where the composition is injected into the middle ear over the area of the target inner-middle ear interface tissue structure, such as the round window niche (see e.g. Light J. and Silverstein H., Current Opinion in Otolaryngology & Head and Neck Surgery 12: 378-383 (2004)). The injection may be performed directly through the tympanic membrane, through a ventilating tube inserted into the tympanic membrane, or through an opening of the tympanic membrane (e.g. by tympanomeatal flap). The volume of the formulation to be injected is typically between about 200 and about 500 microlitres. In particular embodiments, the method of administration to the inner ear is by diffusion across the round window membrane, which is relatively easily accessible from the middle ear space, and allows the inner ear to remain intact, thus avoiding any potential problems from leaking intracochlear fluids. Thus, in some embodiments, the pharmaceutical composition is delivered to the middle ear.

Pharmaceutical compositions which cannot be injected or infused by any of the aforementioned means may be deposited onto the target inner-middle ear interface structure across a small opening in the tympanic membrane with the aid of surgical instrument.

The pharmaceutical composition can be administered prior to, during or after tinnitus has been induced by a cochlear insult as described herein. The amount to be administered may vary, depending upon the method of administration, duration of therapy, the condition of the subject to be treated, the severity of the tinnitus, the particular JNK peptide inhibitor used, and ultimately will be decided by the attending physician. The duration of therapy may range between about one hour and several days, weeks or months, and may extend up to chronic treatment. In the case of therapies of long duration, repeat doses of the pharmaceutical composition may have to be administered.

A therapeutically effective amount or dose is defined as an amount or dose effective to suppress or reduce tinnitus in a treated individual. A therapeutically effective amount or dose is also the amount effective to suppress or reduce tinnitus in the afflicted individual. In one embodiment, a therapeutically effective amount or dose of a JNK peptide inhibitor is an amount or dose effective to reduce the perception of tinnitus by the afflicted individual following administration of the composition. In another embodiment, a therapeutically effective amount or dose of a JNK peptide inhibitor is an amount or dose effective to reduce the loudness of tinnitus following administration of the composition. In another embodiment, a therapeutically effective amount or dose of a JNK peptide inhibitor is an amount or dose effective to reduce the frequency of tinnitus episodes following administration of the composition. In still another embodiment, a therapeutically effective amount or dose of a JNK peptide inhibitor is an amount or dose effective to reduce the duration of tinnitus episodes following administration of the composition.

As stated above, a therapeutically effective amount or dose may vary, depending on the choice of specific JNK peptide inhibitor, the severity of the tinnitus to be treated and on the method of its administration. For example, a lower dose of a JNK peptide inhibitor with a higher binding affinity for JNK may be more effective than a JNK peptide inhibitor that binds with a lower affinity. Additionally, a higher dose of an intravenously administered JNK peptide inhibitor would be required than that of the same pharmaceutical composition administered locally to the round window membrane or oval window of the ear. The therapeutically effective amount or dose of the JNK peptide inhibitor to be delivered may range from about 0.1 mg to about 3 mg, from about 0.2 mg to about 2 mg, or from about 0.3 mg to about 0.8 mg. In certain embodiments, a therapeutically effective amount or dose of a JNK peptide inhibitor comprising or consisting of a sequence of SEQ ID NO: 2 is about 0.2 mg to about 1 mg.

The duration of therapy may also vary, depending on the severity and specific form of tinnitus for which treatment is desired (e.g. acute, subacute, or chronic tinnitus). As a guide, shorter durations of therapy are preferred and are sufficient when the tinnitus does not recur once therapy has ceased. Longer durations of therapy may be employed for an individual in whom the tinnitus persists following short therapy. In one embodiment, a therapeutically effective amount of a JNK peptide inhibitor is administered to the subject in a single dose, for example, a single intratympanic injection. In another embodiment, a therapeutically effective amount of a JNK peptide inhibitor is administered to the subject in multiple doses over the period of days or weeks. In certain embodiments, a therapeutically effective amount of a JNK peptide inhibitor is administered to the subject in multiple intratympanic injections given over the period of three to five consecutive days. For instance, in one embodiment, the subject is administered the JNK peptide inhibitor in three intratympanic injections over three consecutive days with one injection per day.

In some embodiments of the present invention, administration of a JNK peptide inhibitor according to the methods of the present invention provides a statistically significant therapeutic effect. In one embodiment, the statistically significant therapeutic effect is determined based on one or more standards or criteria provided by one or more regulatory agencies in the United States, e.g., FDA or other countries. In other embodiments, the statistically significant therapeutic effect is determined based on results obtained from regulatory agency approved clinical trial set up and/or procedure.

In some embodiments, the statistically significant therapeutic effect is determined based on a patient population of at least 150, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined based on the results of a Phase III clinical trial of the methods provided by the present invention.

In general, statistical analysis can include any suitable method permitted by a regulatory agency, e.g., FDA in the US, EMA in Europe, or regulatory agency in any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM), ANCOVA, and Cox regression analysis.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

### Example 1

### Efficacy of a Peptide Inhibitor of JNK in the Treatment of Inner Ear Tinnitus

c-Jun N-terminal kinase (JNK) is involved in apoptosis of stress injured hair cells and spiral ganglia neurons (Zine et al., 2004; Abi-Hachem et al., 2010), the principal mechanism of cell death in the cochlea following traumatic injury (Hu et al., 2002) or cochlear inflammation (Ma et al., 2000; Barkdull et al., 2007). AM-111 is a 31-amino acid cell-permeable peptide (SEQ ID NO: 2 in which all chiral amino acids are in the D configuration and the peptide is synthesized in the reverse order), formulated in a biocompatible hyaluronic acid gel. The chimeric peptide contains an effector domain derived from the scaffold protein islet-brain 1 which retains JNK in the cytoplasm fused to the Trans-Activator of Transcription (TAT) protein transduction domain (Bonny et al., 2001). Treatment with AM-111 was shown to be otoprotective in various models of cochlear insult: acute noise trauma (Wang et al., 2003; Wang et al. 2007; and Coleman et al. 2007), acute labyrinthitis (Barkdull et al., 2007), aminoglycoside ototoxicity (Wang et al., 2003), bacterial infection (Grindal et al., 2010), cochlear ischemia (Omotehara et al., 2011) and cochlear implantation trauma (Eshraghi et al., 2013). The breadth of AM-111′s therapeutic spectrum suggests a key role of JNK in acute sensorineural hearing loss (ASNHL). However, no statistically significant effect of AM-111 on tinnitus has been previously reported.

A double-blind, randomized, placebo-controlled phase II study was conducted to evaluate the efficacy and safety of AM-111 in treating ASNHL and associated tinnitus. Eligible participants were aged 18 to 60 years and suffered from ASNHL (unilateral idiopathic sudden sensorineural hearing loss (ISSNHL), uni- or bilateral acute acoustic trauma (AAT)) with hearing loss of at least 30 dB and onset not more than 48 hours previously. The hearing loss was determined against a reference value: mean hearing threshold at the 3 most affected contiguous test frequencies (pure tone average, PTA) less corresponding mean hearing threshold of the contralateral ear (Plontke et al., 2007). In case of previously asymmetric hearing or bilateral ASNHL, thresholds from a previous audiogram or ISO-7029; 2000 norm values served as reference. The PTA frequencies determined at baseline remained fixed for all evaluations.

Exclusion criteria included history of Meniere's disease, autoimmune or radiation induced hearing 80 loss, endolymphatic hydrops or fluctuating hearing loss, suspected perilymph fistula, membrane rupture or retrocochlear lesion, barotrauma, air-bone gap >20 dB in 3 contiguous frequencies, previous ASNHL incident within the past 6 weeks, and acute or chronic otitis media or otitis externa. Women who were breast feeding, pregnant or who planned a pregnancy during the study, or women of childbearing potential who declared being unwilling or unable to practice an effective method of 85 contraception were excluded. Written informed consent was obtained from each patient prior to the performance of any study-specific procedures.

At baseline (Day 0), study participants were randomized to receive AM-111 (0.4 or 2.0 mg/mL) or placebo at a 2:1 ratio. The study consisted of a baseline assessment and 4 follow-up visits on Days 3, 7, 30, and 90. Baseline assessments included a general physical examination, vital signs, and a urine pregnancy test for women of childbearing age. At each study visit pure tone hearing thresholds, speech discrimination at 60 and 80 dB, and subjective tinnitus loudness were determined. Those patients reporting tinnitus were asked to rate its loudness “right now” on a numerical scale ranging from 0 (no tinnitus) to 10 (extremely loud).

Approximately 0.25 mL of the study drug was administered on Day 0 by intratympanic (i.t.) injection under local anesthesia through a small myringotomy with the patient's head placed in a position tilted 45° towards the unaffected ear. Patients remained in their reclined or supine position for approximately 30 minutes to allow for diffusion of the active substance into the cochlea. In case of bilateral AAT, only the worst affected ear was treated. Subjects whose PTA recovered <10 dB from baseline to Day 7 were given the option to receive oral prednisolone 50 mg (Ratiopharm, Ulm, Germany) b.i.d. for 5 days. Previous reports showed no difference in outcomes for corticosteroid therapy initiated within the first 24 hours or within the first week (Huy et al., 2005).

The sample size was determined based on an expected effect size of 0.6, a two-sided type I error rate of 5% and a statistical power of 80%. This resulted in a planned sample size of 102 patients per cohort (68 AM-111, 34 placebo), for a total of 204 patients.

Efficacy analyses were primarily performed on a modified “Intention to treat” (mITT) analysis set (treated patients with PTA measured on Day 3±1 or Day 7+4 days at most) and secondarily on the “Per Protocol” (PP) analysis set. The “Safety Population” analysis set included all patients who received an injection of the study medication. Random imputation was performed for missing PTA values at Days 7 and 30, and missing speech discrimination score (SDS) values at Day 7 based on the preceding value and the mean change observed in the respective treatment group (mITT set).

For continuous efficacy endpoints, analysis of covariance (ANCOVA) models were used including treatment group and initial frequency range as class effects, and baseline values of the respective endpoint as covariate. For the complete recovery rate, a logistic regression model was applied including treatment, initial frequency range, and baseline hearing loss as predictor variables. Initial frequency range was included in the models since spontaneous recovery has been reported to be more pronounced in the lower frequencies (Huy et al., 2005). The percentage of subjects with post-treatment remission of ASNHL-related tinnitus was compared using the Fisher exact test.

### Results

A total of 210 patients were screened for and enrolled into the AM-111 phase IIb study in 2 cohorts. Each cohort comprised 105 patients: 70 allocated to AM-111 high dose (2.0 mg/mL) together with 35 allocated to placebo, 68 allocated to AM-111 low dose (0.4 mg/mL) group together with 37 allocated to placebo. All patients received one i.t. injection of study drug and constituted the “Safety Population” analysis set (210 patients). 11 patients (5%) were lost to follow-up and 6 (3%) withdrew consent. A total of 197 patients were included in the modified “Intention to treat” analysis set. The most common reasons for exclusion were study visits not performed within stipulated schedule (5 patients) and taking prohibited medication (5 patients). A total of 188 patients were included in the per protocol analysis set; the most frequent reason for exclusion was violation of the 30 dB minimum hearing loss criterion (8 patients).

The majority of patients were male (61%), suffered from ISSNHL (92%) and had tinnitus as comorbidity (80%). On average, patients were treated 29 hours post ASNHL onset. The mean hearing loss at the 3 most affected test frequencies was 52.2 dB; the mean SDS was 52.3% (60 dB) and 67.6% (80 dB). Clinically relevant spontaneous nystagmus (defined as >5 beats/30 sec) was observed in 7% of patients. Overall, baseline characteristics were similar for treatment groups.

A total of 167 subjects had ASNHL-related tinnitus at baseline (52 subjects who received placebo and 115 subjects who received AM-111). Baseline incidence was 73.2%, 82.4%, and 84.3% in the placebo, AM-111 0.4 mg/mL and AM-111 2.0 mg/mL groups. See Table 1. Incidence of ASNHL-related tinnitus decreased most quickly and most markedly for the AM-111 0.4 mg/mL group (38.0% of affected subjects had ASNHL related tinnitus at D90), followed by the AM-111 2.0 mg/mL group (51.9%) and the placebo group (56.0%). See Table 2.

TABLE 1
Tinnitus at Baseline: Frequencies of Onset with Respect to ASNHL
Onset and Subjective Loudness - Safety Population Analysis Set

| | | AM-111 | AM-111 | |
|---|---|---|---|---|
| | Placebo | 0.4 mg/mL | 2.0 mg/mL | Total |
| | (N = 72) | (N = 68) | (N = 70) | (N = 210) |
| Tinnitus, number | | | | |
| (%) subjects | | | | |
| No | 16 (22.2) | 9 (13.2) | 5 (7.1) | 30 (14.3) |
| Yes | 56 (77.8) | 59 (86.8) | 65 (92.9) | 180 (85.7) |
| Pre-Existing | 4 (5.6) | 3 (4.4) | 6 (8.6) | 13 (6.2) |
| Tinnitus, number | | | | |
| (%) subjects | | | | |
| ASNHL Related | 52 (73.2) | 56 (82.4) | 59 (84.3) | 167 (79.9) |
| Tinnitus, number | | | | |
| (%) subjects | | | | |
| Concurrent with | 50 (70.4) | 55 (80.9) | 54 (77.1) | 159 (76.1) |
| ASNHL | | | | |
| Within 24 hours | 2 (2.8) | 0 | 5 (7.1) | 7 (3.3) |
| after ASNHL | | | | |
| Within 48 hours | 0 | 1 (1.5) | 0 | 1 (0.5) |
| after ASNHL^{a} | | | | |
| Tinnitus Loudness | | | | |
| (ASNHL Related | | | | |
| Tinnitus) | | | | |
| Mean (SD) | 5.6 (1.9) | 5.4 (2.3) | 5.2 (2.2) | 5.4 (2.2) |
| Median | 6.0 | 5.0 | 5.0 | 5.0 |
| Range | 2 to 10 | 1 to 10 | 1 to 10 | 1 to 10 |

N = number of subjects;
ASNHL = acute sensorineural hearing loss;
SD = standard deviation
^{a}Excluding where tinnitus was within 24 hours after ASNHL.

TABLE 2
Summary of Frequencies of Tinnitus in Subjects with ASNHL-Related
Tinnitus - Safety Population Analysis Set

| | Number of Subjects with Tinnitus/Number of Subjects with | | | |
|---|---|---|---|---|
| | Observation (%) | | | |
| | | AM-111 | | |
| | Placebo | 0.4 mg/mL | 2.0 mg/mL | Total |
| Day 0, | 52/52 (100) | 56/56 (100) | 59/59 (100) | 115/115 (100) |
| before study | | | | |
| drug | | | | |
| Day 0, after | 48/52 (92.3) | 53/56 (94.6) | 58/59 (98.3) | 111/115 (96.5) |
| study drug | | | | |
| Day 3 | 49/52 (94.2) | 51/56 (91.1) | 59/59 (100) | 110/115 (95.7) |
| Day 7 | 45/52 (86.5) | 42/55 (76.4) | 51/58 (87.9) | 93/113 (82.3) |
| Day 30 | 32/52 (61.5) | 26/55 (47.3) | 35/56 (62.5) | 61/111 (55.0) |
| Day 90 | 28/50 (56.0) | 19/50 (38.0) | 28/54 (51.9) | 47/104 (45.2) |

Analysis of PTA improvement by hearing loss severity (Jerger et al., 1980) revealed unexpectedly strong spontaneous recovery for lesser severities: by Day 7, placebo-treated patients enrolling with mild to moderate hearing loss (PTA<60 dB; n=41) had recovered already 28.9 dB or 77% of their initial loss, whilst for patients with severe or profound hearing loss (PTA≧60 dB; n=30) it was only 17.3 dB or 24%. ANCOVA revealed a statistically significant interaction term between treatment group and hearing loss severity subgroup (p=0.04), indicating that the latter should be analyzed separately. Mild to moderate hearing loss was essentially fully recovered by Day 90 (just 3 dB or 8.1% remained on average).

In the “severe to profound hearing loss” subgroup, a statistically significantly higher percentage of subjects in the AM-111 0.4 mg/mL group achieved complete tinnitus remission over the 90 days compared with placebo (56.0% vs. 26.1%, p=0.045). A higher percentage of subjects in the AM-111 2.0 mg/mL group also achieved complete tinnitus remission compared with placebo, although this did not reach statistical significance (48.3% vs. 26.1%, p=0.152). In the subgroup “mild to moderate hearing loss”, no statistically significant differences were observed between AM-111 treatment groups and placebo. The results of the efficacy of AM-111 on tinnitus in each of the two hearing loss subgroups are summarized in Table 3 below.

TABLE 3
Frequencies of Subjects with Complete ASNHL-Related Tinnitus
Remission in the Treated Ear, by Treatment Group and
Combined Severity Subgroups - mITT Analysis Set

| Complete Tinnitus Remission | Frequency | Frequency AM-111 | |
|---|---|---|---|
| Time point: Overall | Placebo | 0.4 mg/mL | 2.0 mg/mL |
| All Subjects | N = 51 | N = 50 | N = 56 |
| Frequency (%) | 28 (54.9%) | 35 (70.0%) | 31 (55.4%) |
| p-value placebo - AM-111 | | 0.151 | 1.000 |
| Severe to Profound Subgroup | N = 23 | N = 25 | N = 29 |
| Frequency (%) | 6 (26.1%) | 14 (56.0%) | 14 (48.3%) |
| p-value placebo - AM-111 | | 0.045 | 0.152 |
| Mild to Moderate Subgroup | N = 28 | N = 25 | N = 27 |
| Frequency (%) | 22 (78.6%) | 21 (84.0%) | 17 (63.0%) |
| p-value placebo - AM-111 | | 0.732 | 0.245 |

Comparison of frequency between placebo and active treated groups with Fisher's Exact Test.

The results of the study show that AM-111 appears to be a promising novel approach for treating ASNHL-related tinnitus with a short local therapy, particularly in patients suffering from tinnitus associated with severe to profound acute hearing loss.

All publications, patents and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

Abi-Hachem R N, Zine A, Van De Water T R. The injured cochlea as a target for inflammatory processes, initiation of cell death pathways and application of related otoprotectives strategies. Recent Pat CNS Drug Discov 5(2):147-63, 2010.
Barkdull G C, Hondarrague Y, Meyer T, et al. AM-111 reduces hearing loss in a guinea pig model of acute labyrinthitis. Laryngoscope 117:2174-82, 2007.
Bogoyevitch M A, Ngoei K R W, Zhao T T. c-Jun N-terminal kinase (JNK) signaling: recent advances and challenges. Biochim Biophys Acta 1804:463-75, 2010.
Bogoyevitch, M. A., Kendrick, T. S., Ng, D. C. H. and Ban, R. K. Taking the cell by stealth or storm? Protein transduction domains (PTDs) as versatile vectors for delivery. DNA and Cell Biology 21: 879-894, 2002.
Bonny C, Oberson A, Negri S, et al. Cell-permeable peptide inhibitors of JNK: novel blockers of β-cell death. Diabetes 50:77-82, 2001.
Cardozo A K, Buchillier V, Mathieu M, et al. Cell-permeable peptides induce dose- and length-dependent cytotoxic effects. Biochim Biophys Acta 1768; 2222-34, 2007.
Chan Y. Tinnitus: etiology, classification, characteristics, and treatment. Discov Med 42:133-36, 2009.
Chen C, Halpin C, Rauch S. Oral steroid treatment for sudden sensorineural hearing loss: a ten year retrospective analysis. Otol Neurotol 24:728-33, 2003.
Cima R F F, Maes I H, Joore M A, et al. Specialised treatment based on cognitive behaviour therapy versus usual care for tinnitus: a randomised controlled trial. Lancet 379:1951-59, 2012.
Coleman J K, Littlesunday C, Jackson R, et al. AM-111 protects against permanent hearing loss from acute acoustic trauma. Hear Res 226:70-8, 2007.
Cvorović L, Deric D, Probst R, Hegemann S. Prognostic model for predicting hearing recovery in idiopathic sudden sensorineural hearing loss. Otol Neurotol 29(4):464-9, 2008.
Eshraghi A A, Gupta C, Van De Water T R, et al. Molecular mechanisms involved in cochlear implantation trauma and the protection of hearing and auditory sensory cells by inhibition of c-Jun-N-terminal kinase signalling. Laryngoscope 123(Suppl 1):S1-S14. 2013.
Figueiredo R R, Azevedo A A, Oliveira P M. Correlation analysis of the visual-analogue scale and the Tinnitus Handicap Inventory in tinnitus patients. Revista Brasileira de Otorrinolaringologia 75(1):76-9, 2009.
Fisher, P. M., E. Krausz., D. P. Lane. Cellular delivery of impermeable effector molecules in the form of conjugates with peptides capable of mediating membrane translocation. Bioconjugate Chemistry 12: 825-841, 2001.
Garcia-Echeverria, C, and Ruetz, S. β-Homolysine oligomers: a new class of Trojan carriers. Bioorganic & Medicinal Chemistry Letters 13: 247-251, 2003.
Grindal T C, Sampson E M, Antonelli P J. AM-111 prevents hearing loss from semicircular canal injury in otitis media. Laryngoscope 120:178-82, 2010.
Hall D A, Lainez M J, Newman C W, et al. Treatment options for subjective tinnitus: self reports from a sample of general practitioners and ENT physicians within Europe and the USA. BMC Health Serv Res 11:302, 2011.
Halpin C, Rauch S D. Using audiometric thresholds and word recognition in a treatment study. Otol Neurotol 27(1):110-6, 2006.
Hu B H, Henderson D, Nicotera T M. Involvement of apoptosis in progression of cochlear lesion following exposure to intense noise. Hear Res 166:62-71, 2002.
Huy P T, Sauvaget E. Idiopathic sudden sensorineural hearing loss is not an otologic emergency. Otol Neurotol 26(5):896-902, 2005.
Jastreboff P J. Tinnitus retraining therapy. Progr Brain Res 166:415-23, 2007.
Jerger J, Jerger S. Measurement of hearing in adults. In: Paparella M M, Shumrick D A, eds. Otolaryngology, 2nd edition. Philadelphia: WB Saunders: 1225-49, 1980.
Kamalski D M, Hoekstra C E, van Zanten B G, et al. Measuring disease-specific health-related quality of life to evaluate treatment outcomes in tinnitus patients: a systematic review. Otolaryngol Head Neck Surg 143(2):181-5, 2010.
Labatut T, Daza M J, Alonso A. Intratympanic steroids as primary initial treatment of idiopathic sudden sensorineural hearing loss. Eur Arch Otorhinolaryngol 270(11):2823-32, 2013.
Labus J, Breil J, Stützer H, et al. Meta-analysis for the effect of medical therapy vs. placebo on recovery of idiopathic sudden hearing loss. Laryngoscope 120(9):1863-71, 2010.
Langguth B, Elgoyhen A B. Current pharmacological treatments for tinnitus. Expert Opin Pharmacother 13(17):2495-509, 2012.
Lindsay, M. A. Peptide-mediated cell delivery: application in protein target validation. Current Opinions in Pharmacology 2: 587-594, 2002.
Ma C, Billings P, Harris J P et al. Characterization of an experimentally induced inner ear immune response. Laryngoscope 110:451-56, 2000.
Manning A M, Davis R J. Targeting JNK for therapeutic benefit: from JUNK to gold? Nat Rev Drug Discov 2:554-65, 2003.
Meikle M B, Henry J A, Griest S E, et al. The Tinnitus Functional Index: development of a new clinical measure for chronic, intrusive tinnitus. Ear Hear 33(2):153-76, 2012.
Nosrati-Zarenoe R, Hultcrantz E. Corticosteroid treatment of idiopathic sudden sensorineural hearing loss: randomized triple-blind placebo-controlled trial. Otol Neurotol 33(4):523-31, 2012.
Omotehara Y, Hakuba N, Hato N, et al. Protection against ischemic cochlear damage by intratympanic administration of AM-111. Otol & Neurotol 32(9):1422-7, 2011.
Plontke S K, Bauer M, Meisner C. Comparison of pure-tone audiometry analysis in sudden hearing loss studies: lack of agreement for different outcome measures, Otol Neurotol 28(6):753-63, 2007.
Rauch S D, Halpin C F, Antonelli P J, et al. Oral vs intratympanic corticosteroid therapy for idiopathic sudden sensorineural hearing loss: a randomized trial. JAMA 305(20):2071-9, 2011.
Schramm H M. The role of the osteoimmune axis in the inflammation of the inner auditory ear and with regard to the putative anticarcinogenetic principle: part 2. Inflamm Allergy Drug Targets 9(2):120-9, 2010.
Shargorodsky J, Curhan G C, Farwell W R. Prevalence and characteristics of tinnitus among US adults. Am J Med 123:711-18, 2010.
Stachler R J, Chandrasekhar S S, Archer S M, et al. Clinical practice guideline: sudden hearing loss. Otolaryngol Head Neck Surg 146(3 Suppl):S1-35, 2012.
Stouffer J L, Tyler R S. Characterization of tinnitus by tinnitus patients. J Speech Hear Disord 55(3):439-53, 1990.
Suckfuell M, Canis M, Strieth S, et al. Intratympanic treatment of acute acoustic trauma with a cell-permeable JNK ligand: a prospective randomized phase study. Acta Otolaryngol 127(9):938-42, 2007.
Tan W J T, Thome P R, Vlajkovic S M. Noise-induced cochlear inflammation. World J Otorhinolaryngol 3(3):89-99, 2013.
Tung, C, and Weissleder, R. Arginine containing peptides as delivery vectors. Advanced Drug Delivery Reviews 55: 281-294, 2002.
Tyler R S. Patient preferences and willingness to pay for tinnitus treatments. J Am Acad Audiol 23:115-25, 2012.
Vesterager V. Tinnitus—Investigation and management. BMJ 7082:728-31, 1997.
Wang J, Van De Water T R, Bonny C, et al. A peptide inhibitor of c-Jun N-terminal kinase (D-JNKI-1) protects against both aminoglycoside and acoustic trauma-induced auditory hair cell death and hearing loss. J Neurosci 23:8596-607, 2003.
Wang J, Ruel J, Ladrech S, et al Inhibition of the JNK-mediated mitochondrial cell death pathway restores auditory function in sound exposed animals. Mol Pharmacol 71(3):654-66, 2007.
Zine A, Van de Water T R. The MAPK/JNK signalling pathway offers potential therapeutic targets for the prevention of acquired deafness. Curr Drug Targets CNS Neurol Disord 3(4):325-32, 2004.

1. A method of ameliorating or reducing the occurrence of acute inner ear tinnitus induced by a cochlear insult in a human in need thereof comprising administering to the human a pharmaceutical composition comprising a therapeutically effective amount of a peptide inhibitor of c-Jun N-terminal kinase (JNK) or a pharmaceutically acceptable salt thereof, wherein the peptide inhibitor is no more than 50 amino acids in length and comprises a sequence that is at least 80% identical to the sequence of any one of SEQ ID NOs: 1 to 4 and 13 to 45.

2. The method of claim 1, wherein the peptide inhibitor comprises a sequence that is at least 90% identical to DQSRPVQPFLNLTTPRKPR (SEQ ID NO: 1) or RPKRPTTLNLFPQVPRSQD (SEQ ID NO: 4).

3. The method of claim 1, wherein the peptide inhibitor comprises or consists of the sequence of DQSRPVQPFLNLTTPRKPRPPRRRQRRKKRG (SEQ ID NO: 2) or GRKKRRQRRRPPRPKRPTTLNLFPQVPRSQD (SEQ ID NO: 3).

4. The method of claim 1, wherein all of the chiral amino acids in the peptide inhibitor are in the D configuration.

5. The method of claim 1, wherein all of the chiral amino acids in the peptide inhibitor are in the L configuration.

6. The method of claim 1, wherein the cochlear insult results from acute acoustic trauma, presbycusis, ischemia, anoxia, barotrauma, otitis media, exposure to ototoxic drugs, conductive hearing loss, or sudden deafness.

7. The method of claim 1, wherein the pharmaceutical composition is administered to the human within four weeks following the cochlear insult.

8. The method of claim 1, wherein the pharmaceutical composition is administered to the human within one week following the cochlear insult.

9. The method of claim 1, wherein the pharmaceutical composition is administered to the human within three days following the cochlear insult.

10. The method of claim 1, wherein the human has or is diagnosed with acute hearing loss of at least 60 dB within 48 hours of onset.

11. The method of claim 1, wherein the human has or is diagnosed with acute hearing loss of at least 40 dB that persists after 48 hours of onset.

12. The method of claim 1, wherein the therapeutically effective amount of the peptide inhibitor is effective to reduce the perception of tinnitus following administration of the composition.

13. The method of claim 1, wherein the therapeutically effective amount of the peptide inhibitor is effective to reduce the loudness of tinnitus following administration of the composition.

14. The method of claim 1, wherein the pharmaceutical composition is administered topically via the round window membrane or the oval window membrane to the inner ear.

15. The method of claim 1, wherein the pharmaceutical composition is administered by an intratympanic injection.

16. The method of claim 1, wherein the pharmaceutical composition is delivered to the middle ear.

17. The method of claim 1, wherein the pharmaceutical composition is a gel.

18. The method of claim 1, wherein the pharmaceutical composition comprises about 0.5% to about 1% of hyaluronic acid.

19. The method of claim 1, wherein the pharmaceutical composition comprises a phosphate buffer which buffers the pH of the composition to 6.0 to 7.4.

20. The method of claim 1, wherein the therapeutically effective amount of the peptide inhibitor is about 0.2 mg to about 2 mg.

21. The method of claim 1, wherein the therapeutically effective amount of the peptide inhibitor is about 0.3 mg to about 0.8 mg.

22. The method of claim 1, wherein the therapeutically effective amount of the peptide inhibitor is administered in multiple doses.


SEQUENCE LISTING (45 sequences)

SEQ ID NO: 1 - protein, 19 aa, Artificial Sequence
  synthetic peptide
DQSRPVQPFLNLTTPRKPR

SEQ ID NO: 2 - protein, 31 aa, Artificial Sequence
  chimeric peptide
DQSRPVQPFLNLTTPRKPRPPRRRQRRKKRG

SEQ ID NO: 3 - protein, 31 aa, Artificial Sequence
  chimeric peptide
GRKKRRQRRRPPRPKRPTTLNLFPQVPRSQD

SEQ ID NO: 4 - protein, 19 aa, Artificial Sequence
  synthetic peptide
RPKRPTTLNLFPQVPRSQD

SEQ ID NO: 5 - protein, 12 aa, Human immunodeficiency virus
GRKKRRQRRRPP

SEQ ID NO: 6 - protein, 12 aa, Artificial Sequence
  synthetic peptide
PPRRRQRRKKRG

SEQ ID NO: 7 - protein, 16 aa, Drosophila pseudoobscura
RQIKIWFQNRRMKWKK

SEQ ID NO: 8 - protein, 7 aa, Drosophila pseudoobscura
RRMKWKK

SEQ ID NO: 9 - protein, 18 aa, Artificial Sequence
  SynB1
RGGRLSYSRRRFSTSTGR

SEQ ID NO: 10 - protein, 10 aa, Artificial Sequence
  SynB3
RRLSYSRRRF

SEQ ID NO: 11 - protein, 17 aa, Artificial Sequence
  SynB5
RGGRLAYLRRRWAVLGR

SEQ ID NO: 12 - protein, 8 aa, Artificial Sequence
  polyarginine
RRRRRRRR

SEQ ID NO: 13 - protein, 23 aa, Artificial Sequence
  synthetic peptide
DTYRPKRPTTLNLFPQVPRSQDT

SEQ ID NO: 14 - protein, 35 aa, Artificial Sequence
  synthetic peptide
GRKKRRQRRRPPDTYRPKRPTTLNLFPQVPRSQDT

SEQ ID NO: 15 - protein, 23 aa, Artificial Sequence
  synthetic peptide
TDQSRPVQPFLNLTTPRKPRYTD

SEQ ID NO: 16 - protein, 35 aa, Artificial Sequence
  synthetic peptide
TDQSRPVQPFLNLTTPRKPRYTDPPRRRQRRKKRG

SEQ ID NO: 17 - protein, 18 aa, Artificial Sequence
  synthetic peptide
HKHRPTTLRLTTLGAQDS

SEQ ID NO: 18 - protein, 30 aa, Artificial Sequence
  synthetic peptide
GRKKRRQRRRPPHKHRPTTLRLTTLGAQDS

SEQ ID NO: 19 - protein, 18 aa, Artificial Sequence
  synthetic peptide
SDQAGLTTLRLTTPRHKH

SEQ ID NO: 20 - protein, 30 aa, Artificial Sequence
  synthetic peptide
SDQAGLTTLRLTTPRHKHPPRRRQRRKKRG

SEQ ID NO: 21 - protein, 11 aa, Artificial Sequence
  synthetic peptide
RPKRPTTLNLF

SEQ ID NO: 22 - protein, 23 aa, Artificial Sequence
  synthetic peptide
GRKKRRQRRRPPRPKRPTTLNLF

SEQ ID NO: 23 - protein, 11 aa, Artificial Sequence
  synthetic peptide
FLNLTTPRKPR

SEQ ID NO: 24 - protein, 23 aa, Artificial Sequence
  synthetic peptide
FLNLTTPRKPRPPRRRQRRKKRG

SEQ ID NO: 25 - protein, 11 aa, Artificial Sequence
  synthetic peptide
RPKRPKTLNLF

SEQ ID NO: 26 - protein, 23 aa, Artificial Sequence
  synthetic peptide
GRKKRRQRRRPPRPKRPKTLNLF

SEQ ID NO: 27 - protein, 11 aa, Artificial Sequence
  synthetic peptide
FLNLTKPRKPR

SEQ ID NO: 28 - protein, 23 aa, Artificial Sequence
  synthetic peptide
FLNLTKPRKPRPPRRRQRRKKRG

SEQ ID NO: 29 - protein, 11 aa, Artificial Sequence
  synthetic peptide
RPKRPTFLNLF

SEQ ID NO: 30 - protein, 23 aa, Artificial Sequence
  synthetic peptide
GRKKRRQRRRPPRPKRPTFLNLF

SEQ ID NO: 31 - protein, 11 aa, Artificial Sequence
  synthetic peptide
FLNLFTPRKPR

SEQ ID NO: 32 - protein, 23 aa, Artificial Sequence
  synthetic peptide
FLNLFTPRKPRPPRRRQRRKKRG

SEQ ID NO: 33 - protein, 11 aa, Artificial Sequence
  synthetic peptide
RPKRPTSLNLF

SEQ ID NO: 34 - protein, 23 aa, Artificial Sequence
  synthetic peptide
GRKKRRQRRRPPRPKRPTSLNLF

SEQ ID NO: 35 - protein, 11 aa, Artificial Sequence
  synthetic peptide
FLNLSTPRKPR

SEQ ID NO: 36 - protein, 23 aa, Artificial Sequence
  synthetic peptide
FLNLSTPRKPRPPRRRQRRKKRG

SEQ ID NO: 37 - protein, 11 aa, Artificial Sequence
  synthetic peptide
RPKRPTTLNLD

SEQ ID NO: 38 - protein, 23 aa, Artificial Sequence
  synthetic peptide
GRKKRRQRRRPPRPKRPTTLNLD

SEQ ID NO: 39 - protein, 11 aa, Artificial Sequence
  synthetic peptide
DLNLTTPRKPR

SEQ ID NO: 40 - protein, 23 aa, Artificial Sequence
  synthetic peptide
DLNLTTPRKPRPPRRRQRRKKRG

SEQ ID NO: 41 - protein, 10 aa, Artificial Sequence
  synthetic peptide
PKRPTTLNLF

SEQ ID NO: 42 - protein, 22 aa, Artificial Sequence
  synthetic peptide
GRKKRRQRRRPPPKRPTTLNLF

SEQ ID NO: 43 - protein, 10 aa, Artificial Sequence
  synthetic peptide
FLNLTTPRKP

SEQ ID NO: 44 - protein, 22 aa, Artificial Sequence
  synthetic peptide
FLNLTTPRKPPPRRRQRRKKRG

SEQ ID NO: 45 - protein, 37 aa, Artificial Sequence
  synthetic peptide
YGRKKRRQRRRXILKQSMTLNLADPVGSLKPHLRAKN